SEQUENCE LISTING (8 sequences)

SEQ ID NO: 1 - DNA, 1114 bp, Homo sapiens
attccagatcatagagatgttgaaattggttattattgagaacatggcagaaattatgctattctcattagatctcttgcttttctccacagatatcctttgctttaattttccttctaagatgatcaaacttcctggttttattaccatacaaatcttcttttatccacaagccagctttggaatttcagcaaacaccatccttcttcttttccacatcttcacctttgttttcagtcacaggtctaagtccattgacatgataattagtcacctgtctctcatccacatactgctgctcttcactcaggcaatattggtgtccttagacttctttggttcacagaatactcaggatgatcttaggtataaggtcattgtctttttaaacaaggtgatgaggggcctctccatctgcaccccctgcctcctgagtgtgctccaggccatcatcagccccagcatcttctccttggcaaagctcaaacatccttctgcaagtcacatcttaggattcttccttttctcatgggtcctcaacatgttcattggtgtaatcttctgctgtacactgcggctacccccagtgaaacggggccagtcttctgtttgtcatacagcactgttcctttttgcccatgagctacacccacaggagactgtttttcacactaatgactttgagggatgtcacctttatagggttcatggtcctctcaagaggctacatggtgattattttatacagacaataagaggctatctcagtgccttcacgcagccagcctgtccccgagtctcaccagtgaaaagagcctcccaggctatcttactgctggtgagttttgtcttcacatactgggtggactttacgttctcattttcaggaggtgtgacatggataaatgattctctgctagtgtggctccaggttattgtggccaatagctatgccgcaattagtcctttgatgctaatttatgctgataaccaaatattcaagactctgcaaatgttatggtttaaatatttgtctcctccaaagctcatgttgaaatttaatcgccaatgtggcagtactaagaagtgatgatgagaggttaatccattcatg

SEQ ID NO: 2 - DNA, 1114 bp, Homo sapiens
attccagatcatagagatgttgaaattggttattattgagaacatggcagaaattatgctattctcattagatctcttgcttttctccacagatatcctttgctttaattttccttctaagatgatcaaacttcctggttttattaccatacaaatcttcttttatccacaagccagctttggaatttcagcaaacaccatccttcttcttttccacatcttcacctttgttttcagtcacaggtctaagtccattgacatgataattagtcacctgtctctcatccacatactgctgctcttcactcaggcaatattggtgtccttagacttctttggttcacagaatactcaggatgatcttaggtataaggtcattgtctttttaaacaaggtgatgaggggcctctccatctgcaccccctgcctcctgagtgtgctccaggccatcatcagccccagcatcttctccttggcaaagctcaaacatccttctgcaagtcacatcttaggattcttccttttctcatgggtcctcaacatgttcattggtgtaatcttctgctgtacactgcggctacccccagtgaaacggggccagtcttctgtttgtcatacagcactgttcctttttgcccatgagctacacccacaggagactgtttttcacactaatgactttgagggatgtcacctttatagggttcatggtcctctcaagaggctacatggtgattattttatacagacaataagaggctatctcagtgccttcacacagccagcctgtccccgagtctcaccagtgaaaagagcctcccaggctatcttactgctggtgagttttgtcttcacatactgggtggactttacgttctcattttcaggaggtgtgacatggataaatgattctctgctagtgtggctccaggttattgtggccaatagctatgccgcaattagtcctttgatgctaatttatgctgataaccaaatattcaagactctgcaaatgttatggtttaaatatttgtctcctccaaagctcatgttgaaatttaatcgccaatgtggcagtactaagaagtgatgatgagaggttaatccattcatg

SEQ ID NO: 3 - protein, 357 aa, Homo sapiens
MLKLVIIENMAEIMLFSLDLLLFSTDILCFNFPSKMIKLPGFITIQIFFYPQASFGISANTILLLFHIFTFVFSHRSKSIDMIISHLSLIHILLLFTQAILVSLDFFGSQNTQDDLRYKVIVFLNKVMRGLSICTPCLLSVLQAIISPSIFSLAKLKHPSASHILGFFLFSWVLNMFIGVIFCCTLRLPPVKRGQSSVCHTALFLFAHELHPQETVFHTNDFEGCHLYRVHGPLKRLHGDYFIQTIRGYLSAFTQPACPRVSPVKRASQAILLLVSFVFTYWVDFTFSFSGGVTWINDSLLVWLQVIVANSYAAISPLMLIYADNQIFKTLQMLWFKYLSPPKLMLKFNRQCGSTKK

SEQ ID NO: 4 - protein, 348 aa, Homo sapiens
MAEIMLFSLDLLLFSTDILCFNFPSKMIKLPGFITIQIFFYPQASFGISANTILLLFHIFTFVFSHRSKSIDMIISHLSLIHILLLFTQAILVSLDFFGSQNTQDDLRYKVIVFLNKVMRGLSICTPCLLSVLQAIISPSIFSLAKLKHPSASHILGFFLFSWVLNMFIGVIFCCTLRLPPVKRGQSSVCHTALFLFAHELHPQETVFHTNDFEGCHLYRVHGPLKRLHGDYFIQTIRGYLSAFTQPACPRVSPVKRASQAILLLVSFVFTYWVDFTFSFSGGVTWINDSLLVWLQVIVANSYAAISPLMLIYADNQIFKTLQMLWFKYLSPPKLMLKFNRQCGSTKK

SEQ ID NO: 5 - DNA, 1077 bp, Homo sapiens
atgttgaaattggttattattgagaacatggcagaaattatgctattctcattagatctcttgcttttctccacagatatcctttgctttaattttccttctaagatgatcaaacttcctggttttattaccatataaatcttcttttatccacaagccagctttggaatttcagcaaacaccatccttcttcttttccacatcttcacctttgttttcagtcacaggtctaagtccattgacatgataattagtcacctgtctctcatccacatactgctgctcttcactcaggcaatattggtgtccttagacttctttggttcacagaatactcaggatgatcttaggtataaggtcattgtctttttaaacaaggtgatgaggggcctctccatctgcaccccctgcctcctgagtgtgctccaggccatcatcagccccagcatcttctccttggcaaagctcaaacatccttctgcaagtcacatcttaggattcttccttttctcatgggtcctcaacatgttcattggtgtaatcttctgctgtacactgcggctacccccagtgaaacggggccagtcttctgtttgtcatacagcactgttcctttttgcccatgagctacacccacaggagactgtttttcacactaatgactttgagggatgtcacctttatagggttcatggtcctctcaagaggctacatggtgattattttatacagacaataagaggctatctcagtgccttcacacagccagcctgtccccgagtctcaccagtgaaaagagcctcccaggctatcttactgctggtgagttttgtcttcacatactgggtggactttacgttctcattttcaggaggtgtgacatggataaatgattctctgctagtgtggctccaggttattgtggccaatagctatgccgcaattagtcctttgatgctaatttatgctgataaccaaatattcaagactctgcaaatgttatggtttaaatatttgtctcctccaaagctcatgttgaaatttaatcgccaatgtggcagtactaagaagtgatga

SEQ ID NO: 6 - protein, 45 aa, Homo sapiens
MLKLVIIENMAEIMLFSLDLLLFSTDILCFNFPSKMIKLPGFITI

SEQ ID NO: 7 - DNA, 1071 bp, Homo sapiens
atggcagaaattatgctattctcattagatctcttgcttttctccacagatatcctttgctttaattttccttctaagatgatcaaacttcctggttttattaccatacaaatcttcttttatccacaagccagctttggaatttcagcaaacaccatccttcttcttttccacatcttcacctttgttttcagtcacaggtctaagtccattgacatgataattagtcacctgtctctcatccacatactgctgctcttcactcaggcaatattggtgtccttagacttctttggttcacagaatactcaggatgatcttaggtataaggtcattgtctttttaaacaaggtgatgaggggcctctccatctgcaccccctgcctcctgagtgtgctccaggccatcatcagccccagcatcttctccttggcaaagctcaaacatccttctgcaagtcacatcttaggattcttccttttctcatgggtcctcaacatgttcattggtgtaatcttctgctgtacactgcggctacccccagtgaaacggggccagtcttctgtttgtcatacagcactgttcctttttgcccatgagctacacccacaggagactgtttttcacactaatgactttgagggatgtcacctttatagggttcatggtcctctcaagaggctacatggtgattattttatacagacaataagaggctatctcagtgccttcacgcagccagcctgtccccgagtctcaccagtgaaaagagcctcccaggctatcttactgctggtgagttttgtcttcacatactgggtggactttacgttctcattttcaggaggtgtgacatggataaatgattctctgctagtgtggctccaggttattgtggccaatagctatgccgcaattagtcctttgatgctaatttatgctgataaccaaatattcaagactctgcaaatgttatggtttaaatatttgtctcctccaaagctcatgttgaaatttaatcgccaatgtggcagtactaagaagtgatgatgagaggttaatccattcatg

SEQ ID NO: 8 - DNA, 1071 bp, Homo sapiens
atggcagaaattatgctattctcattagatctcttgcttttctccacagatatcctttgctttaattttccttctaagatgatcaaacttcctggttttattaccatacaaatcttcttttatccacaagccagctttggaatttcagcaaacaccatccttcttcttttccacatcttcacctttgttttcagtcacaggtctaagtccattgacatgataattagtcacctgtctctcatccacatactgctgctcttcactcaggcaatattggtgtccttagacttctttggttcacagaatactcaggatgatcttaggtataaggtcattgtctttttaaacaaggtgatgaggggcctctccatctgcaccccctgcctcctgagtgtgctccaggccatcatcagccccagcatcttctccttggcaaagctcaaacatccttctgcaagtcacatcttaggattcttccttttctcatgggtcctcaacatgttcattggtgtaatcttctgctgtacactgcggctacccccagtgaaacggggccagtcttctgtttgtcatacagcactgttcctttttgcccatgagctacacccacaggagactgtttttcacactaatgactttgagggatgtcacctttatagggttcatggtcctctcaagaggctacatggtgattattttatacagacaataagaggctatctcagtgccttcacacagccagcctgtccccgagtctcaccagtgaaaagagcctcccaggctatcttactgctggtgagttttgtcttcacatactgggtggactttacgttctcattttcaggaggtgtgacatggataaatgattctctgctagtgtggctccaggttattgtggccaatagctatgccgcaattagtcctttgatgctaatttatgctgataaccaaatattcaagactctgcaaatgttatggtttaaatatttgtctcctccaaagctcatgttgaaatttaatcgccaatgtggcagtactaagaagtgatgatgagaggttaatccattcatg

(19) United States
(12) Patent Application Publication
(10) Pub. No.: US 2004/0067887 A1
(43) Pub. Date: Apr. 8, 2004

(54) Human VNO receptor (R1)

(76) Inventors: Jian Zhang, San Mateo, CA (US); Ronald C. Herman, Sunnyvale, CA (US)

Correspondence Address: HELLER EHRMAN WHITE & MCAULIFFE LLP, 275 MIDDLEFIELD ROAD, MENLO PARK, CA, 94025-3506, US

(21) Appl. No.: 10/697,147

(22) Filed: Oct. 29, 2003

Related U.S. Application Data
(63) Continuation of application No. 09/728,309, filed on Nov. 30, 2000.

(51) Int. Cl. A61K 38/17; C07K 14/705

(52) U.S. Cl. 514/12; 530/350; 536/23.5; 435/69.1; 435/320.1; 435/325

(57) ABSTRACT

This invention relates to a human pheromone receptor. Specifically, a human VNO receptor and its mRNA, cDNA and amino acid sequence are disclosed. Expression vectors comprising a nucleotide sequence encoding a human pheromone receptor are contemplated. Cells may be transformed with said expression vectors. Cells expressing the pheromone receptor in vitro may be utilized to screen drug candidates to identify agonists and/or antagonists of receptor activation. Drugs so identified find a therapeutic use in psychiatric disorders and neuroendocrine disorders and diseases.

### FIELD

The present invention relates generally to the field of receptors, specifically pheromone receptors, and more specifically to human vomeronasal organ receptors.

### BACKGROUND

Small, volatile and non-volatile organic molecules, commonly referred to as pheromones, mediate species-specific chemical communication between terrestrial animals. Pheromones are present in the secretions and excretions from various organs and tissues, including the skin, and represent diverse families of chemical structures. Pheromones play essential roles in sexual activity, reproductive biology, and other innate animal behaviors (Luscher et al., (1959) Nature 18:55-56; Meredith (1983) in Pheromones and Reproduction in Mammals (Vandenbergh, ed.) pp. 199-252, Academic Press; Stern et al., (1998) Nature 392:177-179; Wysocki, (1979) Neurosci. Biobehav. Rev. 3:301-341; Jacob et al., (2000) Hormones and Behavior 37:57-78; Grosser et al., (2000) Psychoneuroendocrinology 25:289-299). Some, but not all, terrestrial vertebrates detect pheromones in the vomeronasal organ (the VNO), also known as Jacobson's organ, a small dead-end tubular structure with an opening into the nasal cavity that is located bilaterally at the base of the nasal septum (Moran et al., (1991) J. Steroid Biochem. Molec. Biol. 39:545-552.).

The VNO was first identified in humans in 1703 but it was believed to be a vestigial organ without function in the adult. In the 1990s, the presence of a VNO was established, caudal to the nasal septal cartilage on both sides of the nasal septum, in more than 1700 normal male and female human subjects (Berliner, (1996) J. Steroid Biochem. Molec. Biol. 58:1-2; Gaafar et al., (1998) Acta Otolargyngol. 118:408-412; Smith et al., (1998) Micro. Res. Tech. 41:483-491) The VNO is physically separate and functionally distinct from the olfactory epithelium that detects the volatile odorants. Odorants do not bind to the VNO receptors.

The VNO is lined with neuroepithelial cells with a microvillar surface that is the presumptive site of pheromone receptors. Immunohistochemical staining of adult human VNO epithelium detects neuron-specific enolase and protein gene product (PGP) 9.5, both neuronal and neuroendocrine markers, in some bipolar cells with morphological similarities to olfactory receptor neurons (Takami et al., (1993) Neuroreport 4:375-378). More recent studies show that the majority of the cells lining the lumen of the human VNO stain with antibodies to synaptophysin or chromogranin which are also markers for neuronal and neuroendocrine cells. These data provide clear evidence for the existence of a neuroepithelium in the human VNO. However, Takami et al. (1993) do not detect olfactory marker protein (OMP) in the human VNO even though it is expressed in the VNO of other vertebrates including rodents. This may reflect an important and interesting species difference between humans and other vertebrates.

In animals, signals from the olfactory epithelium travel via the olfactory bulb to the olfactory cortex and then on to other regions of the brain. In contrast, signals from the VNO are transmitted through the accessory olfactory bulb to the amygdala and hypothalamus (Broadwell et al., (1975) J. Comp. Neurol. 163:329-346; Kevetter et al., (1981) J. Comp. Neurol. 197:81-98). Surgical ablation of the VNO in male rodents alters a variety of endocrine-mediated responses to female pheromones including androgen surges, vocalization, territorial marking, and inter-male aggression. Ablation of the VNO in female rodents delays or prevents activation of reproduction, abolishes the effects of over-crowding on sexual maturation, and reduces maternal responses to intruders (Wysocki et al., (1991) J. Steroid Biochem. Molec. Biol. 39:661-669). In humans, the defect(s) that causes the inherited hypogonadal disorder, Kallmann Syndrome, is also associated with defective development of the VNO-terminalis complex (Kallmann et al., (1943) Am. J. Ment. Defic. 48:203-236).

Application of only femtomole quantities of any of several proprietary, synthetic vomeropherins directly to the VNO of human volunteers rapidly induces reproducible negative voltage potentials that can be measured locally with a multifunctional miniprobe. The electrophysiological response in the VNO is characteristic of a mass receptor potential. The magnitude of the response is dose-dependent and is accompanied by changes in autonomic nervous system function, brain wave activity, gonadotropin secretion, and mood (Berliner et al., (1996) J Steroid Biochem, Molec. Biol. 58:259-265; (1998a) J. Steroid Biochem. Molec. Biol. 65:237-242; Monti-Bloch et al., (1998b) Ann. N.Y. Acad. Sci. 855:373-389; Monti-Bloch et al., (1994) Pyschoneuroendocrinology 19:673-686; Monti-Bloch et al., (1991) J. Steroid Biochem. Molec. Biol. 39:573-582; Grosser et al., (2000) Psychoneuroendocrinology 25:289-299).

Recent fMRI studies detect dose-dependent activation of the anterior medial thalamus, the inferior frontal gyrus, and other regions of the human brain, in the absence of detectable odor, following administration of estra-1,3,5(10),16-tetraen-3yl acetate (PH15) to human volunteers. Although Sobel et al. ((1999) Brain 122:209-217) deliver the compound non-specifically to the nasal cavity in these fMRI tests, Monti-Bloch et al. (1994) have demonstrated that this compound induces physiological responses in vivo only when applied specifically to the VNO but not when applied to either olfactory or respiratory epithelium of human subjects. Therefore, the fMRI data support the existence of a functional neurological connection between the VNO and the human brain which can be activated by a vomeropherin.

Administration of naturally occurring compounds of known structure such as estra-1,3,5(10),16-tetraen-3-ol and androsta-4,16-dien-3-one to the human VNO induce bradycardia, bradypnea, increases in core body temperature, and other physiological responses. Stern et al. (1998) have demonstrated that odorless human pheromones, obtained from the axillae of women at different stages of the menstrual cycle, exert opposing effects on ovulation when applied above the lips where they can volatilize into the nasal cavity of recipient females. Some vomeropherins act exclusively in human females or in males, and others exert opposite effects on autonomic reflexes such as body temperature. Taken together, these data provide substantial support for the existence of a functional VNO in humans with the capacity to exert significant physiological effects in vivo.

The VNO system affords the unique opportunity to develop and market novel therapeutics to treat disease via previously unexploited targets and neurological pathways. This approach has substantial benefits for the patient over existing therapies including: (i) the ease of delivery to the VNO, (ii) the requirement for only picograms of drug, (iii) the rapid response to drug, and (iv) the apparent absence of the side-effects and toxicity frequently associated with systemic (e.g., oral) delivery of drug. Thus, targeting receptors in the human VNO for the treatment of disease is desirable.

The standard bioassay for screening candidate vomeropherins requires the participation of human volunteers because pheromones are species-specific. In this assay, the compounds are delivered directly to the VNO of volunteers under IRB-approved protocols, thus necessitating prior toxicological study of each candidate vomeropherin in rodents. This expensive and time-consuming process limits the number of compounds that can be tested and hampers the detailed structure-activity relationship (SAR) analyses that are essential to successful drug discovery.

Viable neuroepithelial cells may be harvested directly from the human VNO for testing in vitro. The harvested VNO cells retain their characteristic neuroepithelial morphology in culture and respond electrophysiologically to the application of vomeropherins in vitro, thereby demonstrating the existence of functional receptors in cells from the target tissue. Although this method still requires the participation of human volunteers, it increases the screening throughput and decreases the number of animals required for toxicological studies. However, only a limited number of non-dividing cells with a ˜2-week life-span are obtained from each volunteer, and thus we require an entirely new approach to meet the demands of modern high throughput drug screening and SAR.

Several groups have cloned receptor cDNAs that are expressed exclusively in the VNO of rats and mice, but, to date, no one has cloned human VNO receptor cDNAs. The sequence of the cloned rodent receptor cDNAs indicates that they belong to the superfamily of G protein-coupled receptors containing seven transmembrane domains, but they are unrelated to any of the G protein-coupled receptors expressed in the olfactory epithelium (Dulac et al., (1995) Cell 83:495-206; Herrada et al., (1997) Cell 90:763-773; Matsunami et al., (1997) Cell 90:775-784; Ryba et al., (1997) Neuron 19:371-379; Saito et al., (1998) Brain Res. Molec. Brain Res. 60:215-227). Database comparisons identify motifs common to Ca²⁺-sensing and metabotropic glutamate receptors in some of the clones. The apparent lack of homology to olfactory receptors is consistent with the observation that many vomeropherins are inactive when applied specifically to human olfactory epithelium in vivo.

Each cloned rodent receptor messenger RNA (mRNA) is detected by in situ hybridization in only a small number of neuroepithelial cells that are dispersed throughout the rodent VNO, and it is likely that each cell expresses only a single receptor gene. (Dulac et al., 1995; Herrada et al., 1997; Matsunami et al., 1997; Ryba et al., 1997; Saito et al., 1998). Some of the cloned rodent receptors exhibit sexually dimorphic expression, i.e., they are expressed differently in males or females.

The rodent VNO receptors are assigned to separate multi-gene families by two criteria: (i) the length of the extracellular (N-terminal) protein domain, and (ii) the isoform of the signal-transducing G protein co-expressed in the same cell. Receptors in the “V1R” family have a relatively short extracellular N-terminal domain and are expressed primarily in cells that express a Gαᵢ isoform of G protein. Receptors in the “V2R” family have a long extracellular N-terminal domain and are expressed primarily in cells that express a Gα₀ isoform of G protein. Differences at the N-terminus between the V1R and V2R families may reflect differences in the structure of the ligand and/or in the location of the ligand-binding domain. (Matsunami et al., 1997; Ryba et al., 1997; Krieger et al., (1999 J. Biol. Chem. 274:4656-4662). Neuroepithelial cells expressing these distinct G protein isoforms are spatially segregated in the VNO in separate apical and basal longitudinal zones, suggesting that there is true physiological significance to the differences between the V1R and V2R receptor families.

Krieger et al. (1999) have recently shown that G protein-coupled receptors expressed in the rodent VNO are functionally linked to signal transduction pathways. Their results demonstrate that volatile and non-volatile pheromonal components of male rat urine selectively activate the major Gα protein subtypes (Gᵢ and G₀, respectively) expressed in the VNO of female rats. The data imply that V1R family receptors, which are co-expressed with Gᵢ, respond to volatile compounds whereas V2R family receptors, which are co-expressed with G₀, respond to non-volatile protein components of urine.

Dulac and Axel (1995) estimate that, in total, the rat V1R family contains approximately 35 candidate pheromone receptors; Herrada and Dulac (1997) and Ryba and Tirindelli (1997) estimate that the rat V2R family contains an additional 100 receptors. Of the various rodent tissues tested, only mRNA from the VNO gives a positive signal on northern blots probed with the cloned (³²P-labeled) pheromone receptor cDNAs. At this limit of sensitivity, these results suggest that the pheromone receptors are expressed exclusively (primarily) in the VNO. At the present time, it is not known if each VNO receptor recognizes a distinct pheromone or if several receptors recognize the same compound.

At reduced stringency, the cloned rodent VNO receptor cDNAs cross-hybridize to human genomic DNA. Dulac and Axel (1995) detect approximately 15 human genes that cross-hybridize to rat V1R family probes, and Herrada and Dulac (1997) detect an additional ten human homologues that cross-hybridize to rat V2R family probes. The two sequenced human V1R genomic DNA clones have ˜40-50% identity with the closest rat homologue. However, both human genomic clones have a stop codon in the putative coding region and may thus be pseudogenes (Dulac and Axel, 1995). Nevertheless, cross-hybridization suggests the evolutionary conservation of G protein-coupled receptors in the VNO and thereby provides a means to isolate human receptor clones.

The presence of these pseudogenes does not preclude the existence of functional human VNO receptor genes, especially in view of our assays with cells harvested directly from the VNO (Monti-Bloch (1997) Chemical Senses 22:752). The past difficulties in isolating, characterizing and cloning a VNO receptor reinforce our assertion that an appropriate way to isolate functional clones of the human VNO receptors is via cDNA prepared directly from the target tissue. In fact, Cao et al. ((1998) Proc. Natl. Acad. Sci. USA 95:11987-11992) have successfully isolated homologues from a goldfish cDNA library using probes based on the rodent receptor sequences even though that species lacks a defined VNO. The presence of pseudogenes in the family has not prevented the successful cloning of olfactory or VNO receptors from a variety of species and they should present no greater obstacle to the cloning of human VNO receptors.

Rodriguez et al. (Nature Genetics 26:18-19; 2000) using a human genomic library, detected a single gene expressed in human olfactory mucosa that is alleged to be a homologue of the rodent V1R sequences. They failed, however, to demonstrate the expression of the receptor in the VNO.

Thus, isolation and characterization of the human VNO receptors is desirable for the development of new drugs, high throughput assays and characterization of the receptors and their signal transduction pathways.

### SUMMARY

The present invention is directed to a human pheromone receptor.

In one aspect of the invention there is a mRNA sequence encoding a human VNO receptor or a portion thereof.

In a second aspect of the invention there is provided a human VNO receptor cDNA sequence or a portion thereof. In one embodiment, there is a DNA sequence encoding a functional receptor. In a second embodiment there is a DNA sequence encoding a mutant allele.

In an another aspect there is a vector comprising the human VNO receptor cDNA sequence encoding a functional pheromone receptor. The vector is capable of directing expression of the encoded receptor in an appropriate cell or cell line.

In a further aspect there is provided transformed cells expressing a functional human VNO receptor. In a preferred embodiment, the cells are oocytes such as human, mouse, xenopus, etc. transformed or injected with the expression vector.

In another aspect of the invention there is provided a cell culture expressing a functional pheromone receptor.

In yet another aspect there is provided a high throughput drug screening assay.

In a further aspect there are provided ligands that bind a human VNO receptor. The ligands may be either agonists or antagonists, naturally-occurring or synthetic. The ligand may find use in the treatment of depression, anxiety, phobias, blood pressure, pain, pre-menstrual syndrome, endocrine disorders, sleep disorders, alertness, and sexual desire

In yet a further aspect of the invention there is provided antibodies to the human VNO receptor.

In another further aspect of the invention there is provided antisense oligonucleotide sequences capable of binding to and blocking the expression of the human VNO receptor. In a preferred embodiment the oligonucleotide binds the mRNA encoding the human VNO receptor. In another embodiment the oligonucleotide binds the DNA encoding the human VNO receptor.

In another aspect, there is provided a transgenic animal expressing the human pheromone receptor. In another aspect there is a second allele of the gene for the receptor that contains both a termination codon which, if expressed, would encode a truncated form of the receptor, and a silent mutation which, if expressed, would not change the encoded amino acid. In another aspect, there is a genetic test that will determine if a subject will benefit from activation of the receptor, inhibition of the receptor or gene therapy with a vector expressing the receptor.

In another aspect, there is provided an oligonucleotide probe capable of hybridizing to and detecting RNA or DNA sequences that encode the receptor, or under reduced stringency hybridization conditions, detect related sequences. Such related receptor sequences may share 30%, 50% or a higher level of homology. In a final aspect, there is an oligonucleotide probe capable of distinguishing between the functional and mutant alleles of the human VNO receptor.

### DESCRIPTION OF THE FIGURES

FIG. 1 is the nucleotide sequence of hVNO-R1.

FIG. 2 is the long form amino acid sequence of hVNO-R1. This form of hNVOR-1 is translated using the first in-frame start codon.

FIG. 3 is the short form amino acid sequence of hVNO-R1. This form of hNVOR-1 is translated using the second in-frame start codon.

FIG. 4 is the long form amino acid sequence of hVNO-R1 with the seven theoretical transmembrane domains indicated.

FIG. 5 is the nucleotide sequence of the mutant hVNO-R1 allele.

### DETAILED DESCRIPTION

The invention will now be described in detail by way of reference only using the following definitions and examples. All patents and publications referred to herein are expressly incorporated by reference.

The present invention provides a human pheromone receptor. The receptor was cloned from human female VNO-specific mRNA and identified in a VNO-specific cDNA library, which are unique resources for the identification and isolation of genes expressed in the VNO, specifically genes for pheromone receptors, ion channels and prospective reagents for high throughput assays. Although the human female VNO has been used and is described in detail herein, the male VNO may be subjected to the same methods and procedures to yield similar mRNA and a cDNA library. Thus, identification and characterization of pheromone receptors, as well as the sexually dimorphic pheromone response, may be investigated. Additionally, cells expressing the pheromone receptor in vitro may be utilized to screen drug candidates to identify agonists and/or antagonists. Drugs so identified find a therapeutic use in psychiatric disorders and neuroendocrine disorders and diseases. Those diseases and disorders include, but are not limited to, premenstrual syndrome (PMS), premenstrual dysphoric disorder (PMDD), depression, anxiety, alertness, sleep disorders, weight and appetite control, fertility, contraception, hormone regulation and the like.

Definitions

As used herein, the following terms or abbreviations, whether used in the singular or plural, will have the meanings indicated:

“Antibody” means an immunoglobulin that specifically binds to and is thereby defined as complementary with a particular spatial and polar organization of another molecule such as a human pheromone receptor. The antibody can be monoclonal or polyclonal and can be prepared by techniques that are well known in the art such as immunization of a host and collection of sera (polyclonal) or by preparing continuous hybridoma cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgGI, IgG2a, IgG2b and IgG3, IgM, etc. Fragments thereof may include Fab, Fv and F(ab′), Fab′, and the like. In addition, aggregates, polymers, and conjugates of immunoglobulins or their fragments can be used where appropriate so long as binding affinity for a particular molecule is maintained.

An “expression construct” or “expression vector” is a vector which is constructed so that the particular coding sequence is located in the vector with the appropriate control sequences including regulatory sequences, the positioning and orientation of the coding sequence with respect to the control sequences being such that the coding sequence is transcribed under the “control” of the control or regulatory sequences. Modification of the sequences encoding the particular protein of interest may be desirable to achieve this end. For example, in some cases it may be necessary to modify the sequence so that it may be attached to the control sequences with the appropriate orientation; or to maintain the reading frame or to target the encoded protein to the correct subcellular location such as the plasma membrane. The control sequences and other regulatory sequences may be ligated to the coding sequence prior to insertion into a vector. Alternatively, the coding sequence can be cloned directly into an expression vector which already contains the control sequences and an appropriate restriction site which is in reading frame with and under regulatory control of the control sequences.

A “ligand” is a molecule that binds to another molecule, used especially to refer to a small molecule that binds specifically to a larger molecule, e.g., an antigen binding to an antibody, a hormone or neurotransmitter binding to a receptor, or a substrate or allosteric effector binding to an enzyme. Ligand may be used interchangably with agent.

A “pheromone” is a biochemical produced by an animal or individual which elicits a specific physiological or behavioral response in another member of the same species. In addition to physiological responses, pheromones can be identified by their species specific binding to receptors in the vomeronasal organ (VNO). Thus, human pheromones bind to human receptors. This can be demonstrated by measuring the change in the summated potential of neuroepithelial tissue in the presence of the pheromone. Human pheromones induce a change of at least about −5 millivolts in human neuroepithelial tissue of the appropriate sex (The binding of pheromones is generally sexually dimorphic.). Naturally occurring human pheromones induce sexually dimorphic changes in receptor binding potential in vivo in the human VNO. Naturally occurring human pheromones can be extracted and purified from human skin and they can also be synthesized. “Human pheromones” are pheromones that are naturally occurring in humans and effective as a specifically binding ligand in human VNO tissue, regardless of how the pheromone was obtained. Thus, both a synthesized and purified molecule may be considered a human pheromone. Commonly, pheromones affect development, reproduction, mood, and related behaviors.

As used herein, an oligonucleotide probe is a DNA or RNA fragment that includes a sufficient number of nucleotides to hybridize specifically to DNA or RNA that includes identical or closely related sequences of nucleotides. A probe may contain any number of nucleotides, from as few as 7 and as many as is desired. The conditions and protocols for such hybridization reactions are well-known to those of skill in the art as are the effects of probe size, temperature, degree of mismatch, salt concentration and other parameters on the hybridization reaction. For example, the lower the temperature and higher the salt concentration at which the hybridization reaction is carried out, the greater the degree of mismatch that may be present in the hybrid molecules.

To be used as a hybridzation probe, the nucleic acid is generally rendered detectable by labeling it with a detectable moiety or label, such as ³⁵S, ³²P, ³H and ¹⁴C, or by other means, including chemical labeling, such as by nick-translation in the presence of deoxyuridylate biotinylated at the 5′-position of the uracil moiety. The resulting probe has incorporated the biotinylated uridylate in place of thymidylate and can be detected based on the binding of streptavidin to the biotin moiety. Any other label may be used as long as it renders the probes sufficiently detectable.

For the purposes herein, the following stringency conditions are defined:

1) high stringency: 0.1×SSPE (1×SSPE=150 mM NaCl, 10 mM NaH₂PO₄, pH 7.4, 1 mM EDTA), 0.1% SDS, 65° C.

2) medium stringency: 0.2×SSPE, 0.1% SDS, 50° C.

3) low stringency: 1.0×SSPE, 0.1% SDS, 50° C.

or any other combination of salt and temperature and other reagents that result in selection of the same degree of mismatch or matching.

“Sexually dimorphic” refers to a difference in the effect of, or response to, a compound or composition between males and females of the same species.

“Vomeropherin” as used herein is a more general term which includes pheromones and describes a substance from any source which functions as a chemosensory messenger, binds to a specific vomeronasal neuroepithelial receptor, and induces a physiological or behavioral effect. The physiologic effect of a “vomeropherin” is mediated through the vomeronasal organ. Vomeropherins may be naturally occurring compounds, synthetic modifications of natural compounds or totally synthetic compounds.

The terms “vector” or “plasmid” as used herein mean a polynucleotide comprised of single strand, double strand, circular, or supercoiled DNA or RNA capable of replicating in a host cell and into which a coding sequence can be inserted. A typical vector may be comprised of the following elements operatively linked at appropriate distances for allowing functional gene expression: replication origin, promoter, enhancer, 5′ mRNA leader sequence, ribosomal binding site, nucleic acid cassette, termination and polyadenylation sites, and selectable marker sequences. One or more of these elements may be omitted in specific applications. The nucleic acid cassette can include a restriction site for insertion of the nucleic acid sequence to be expressed. In a functional vector the nucleic acid cassette contains the nucleic acid sequence to be expressed including translation initiation and termination sites. An intron optionally may be included in the construct, preferably ≧100 bp 5′ to the coding sequence. Vector and plasmid are used interchangeably herein.

Cultured VNO cells are of limited value as a screening tool due to the need to continually isolate new cells. Thus, it would be advantageous to clone and express pheromone receptors in a cell line.

Normal human female and male VNO specimens were obtained by a team of surgeons from donors, with informed consent, undergoing a surgical procedure necessary to treat an injury, disease or other condition of the nasal septum. RNA was isolated from a pool of female VNOs or from individual female and male VNO specimens. Human VNO RNA is essential because (i) the receptors are species-specific, (ii) the receptors may be expressed exclusively in the VNO, and (iii) human genomic DNA contains receptor pseudogenes and introns.

The pooled female VNO RNA was used to prepare a cDNA library. In brief, RNA was extracted from pooled VNO specimens and reverse transcribed with SUPERSCRIPT II reverse transcriptase (Life Technologies) to make first-strand cDNA using a Not I-oligo(dT)₁₂₋₁₈ primer. E. coli DNA polymerase and RNase H were used for second-strand synthesis. Sal I adapters were ligated to the ends and the double-stranded cDNA was digested with Sal I and Not I. The cDNA was directionally ligated into pCMV-Sport7.neo (Life Technologies) and transformed into E. coli.

Certain vomeropherins elicit sexually dimorphic responses and some of the receptors are expressed dimorphically. In consideration of these observations, we constructed our VNO cDNA library with tissue obtained exclusively from human females. Although others have successfully prepared cDNA libraries from individual rodent VNO neuroepithelial cells, we used whole VNO tissue pooled from a number of donors in order to maximize the number, size, and diversity of receptor clones in our library. The library provides an excellent source to search for novel genes, gene fragments, or other nucleotide sequences encoding proteins that are implicated in detection of pheromones or other vomeropherins in the human VNO. Plasmid vectors are currently available that can accommodate the directional cloning of cDNA such that T7 and SP6 RNA polymerase promoter sequences can be used to generate sense and antisense transcripts for subtractive hybridization and riboprobe synthesis.

The GenBank human high throughput genome sequence (htgs) database was searched for potential open reading frames capable of encoding a protein with homology to one or more of the putative rodent pheromone receptors. Expression of the inventive receptor nucleotide sequence in the human VNO was determined by a combination of reverse transcription and polymerase chain reaction (RT-PCR) using RNA from individual female and male VNO specimens and using cloned cDNA from a human female VNO cDNA library.

The VNO RNA was reverse transcribed into first-strand complementary DNA (cDNA) using AMV reverse transcriptase and a mixture of random hexamer primers. The first-strand cDNA was used as the template in PCR reactions that were primed with oligonucleotide pairs whose sequence was based on the targets identified in GenBank. Positive results (i.e., synthesis and detection of an amplicon of the predicted size) were obtained with both female and male VNO cDNA using the primer pair designed to amplify a portion of the sequence designated hVNO-R1. PCR analyses with this primer pair demonstrated that the target sequence was also represented in the female VNO cDNA library. The entire predicted hVNO-R1 sequence was subsequently amplified using the VNO first-strand cDNA as template by PCR with primers flanking the entire open reading frame.

Screening techniques other than PCR, RT-PCR or hybridization are well known to those of skill in the art and the selection of the techniques does not limit the present invention. The procedures for isolating and identifying gene fragments are well known to those of skill in the art; see, e.g. T. Maniatis et al, Molecular Cloning (A Laboratory Manual), Cold Spring Harbor Laboratory Press.

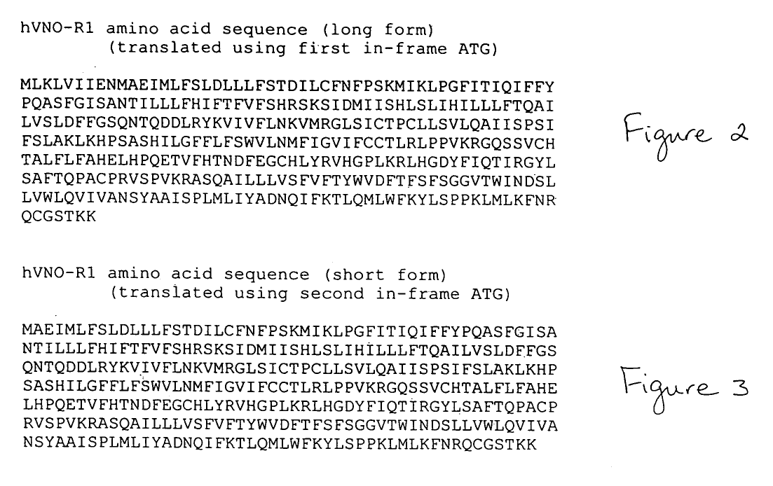

The amplified DNA containing the complete open reading frame was cloned and sent to Sequetech (Mountain View, Calif.) for sequencing on a standard fee-for-service basis. The sequence of the receptor clone hVNO-R1 (SEQ ID No. 1) differs from the corresponding GenBank human genomic DNA entry, and from a second hVNO-R1 clone (designated hVNO-R1 Gen; SEQ ID No. 2), by only a single nucleotide difference (G→A) at position 778. Nevertheless, both sequences encode the same protein due to the known degeneracy of the genetic code. The nucleotide sequence (SEQ ID No. 1) has a start codon beginning at position 17 which predicts a protein with the amino acid sequence shown in FIG. 2 (SEQ ID No. 3). Alignment of the amino acid sequence with putative rodent pheromone receptors indicates that the closest rodent homologue may be the rat VN6 receptor. The two protein sequences share 46% homology when conservative amino acid changes are permitted. A second in-frame start codon at position 44 would give rise to a shorter version of the receptor (SEQ ID No. 4).

Alignment of hVNO-R1 (SEQ ID. No. 1) with the human V-1RL1 sequence (Rodriguez et al; 2000) shows that the two proteins share 37% homology when conservative amino acid changes are permitted. Thus, hVNO-R1 is a novel receptor with some homology to other putative rodent and human pheromone receptors.

A second allele of the VNO receptor sequence (denoted as PP166; FIG. 5; SEQ ID No. 5) is also expressed as an RNA in the VNO of some subjects but differs from hVNO-R1 (SEQ ID No. 1). A point mutation (C→T) at position 136 of SEQ ID No. 5 creates a termination codon, which, if translated into a protein, will yield a truncated form of the receptor (SEQ ID No.6). The silent base difference (G→A) is also present at position 762 of SEQ ID No. 5 but, due to the degeneracy of the genetic code, will not alter the encoded amino acid if the sequence were expressed as a protein.

The existence of two alleles of the receptor sequence enables a genetic test to determine whether a human subject will have a physiological response to administration of either an agonist or antagonist ligand. Subjects who express only the alternative form of the mRNA may not be expected to respond to administration of the ligand. Such subjects may not experience a therapeutic benefit from the compound and could be excluded from such trials and treatments and/or become candidates for gene therapy using a vector that expresses a functional form of the receptor.

Once identified and sequenced, the nucleotide fragments of the genes of the invention may be readily synthesized by conventional means such as solid phase oligo-DNA synthesis (Letsinger et al., (1965) Oligonucleotide synthesis on a polymer support. J. Am. Chem. Soc. 87:3526-3227). Alternatively, the DNA may be produced by recombinant methods, then sequenced. Cloning procedures are conventional and are described by T. Maniatis et al, Molecular Cloning (A Laboratory Manual), Cold Spring Harbor Laboratory (1982). Further, hybridization or PCR methods can be performed using known probes in order to determine whether or not a selected gene is expressed in a gender specific manner may be performed.

Cloned probes which can be made starting from any DNA fragment according to this invention, and thus to recombinant DNAs containing such fragments, are also contemplated herein. Using the cloned DNA fragments as a molecular hybridization probe—either by labeling with radionucleotides or with fluorescent reagents—differences in the expression of the two alleles may be detected.

Oligonucleotide probes based on the VNO receptor gene sequence (SEQ ID No. 1) capable of hybridizing to and detecting RNA or DNA sequences are also encompassed by the present invention. Under stringent conditions the probes detect RNA or DNA sequences encoding the receptor, or under reduced stringency hybridization conditions, the oligonucleotide probes detect related sequences. Such related receptor sequences may share a level of homology of preferably about 30%, more preferably about 50% or most preferably about 75%.

Probes according to the invention can also be used for rapid screening of nucleic acids derived from the tissue of patients, to see if the receptor DNA or RNA is present in the patient's VNO and other tissues. An oligonucleotide probe capable of distinguishing between the functional and mutant alleles of the human VNO receptor may also be used as a probe. These oligonucleotide sequences comprise the region surrounding the point mutation at position 136 of SEQ ID No. 5 described herein.

A method which can be used for such screening comprising the following steps: extraction of DNA from tissue, restriction enzyme cleavage of said DNA, electrophoresis of the fragments and detection of single DNA base mutations with mismatch repair enzymes (Lu et al., Genomics 14:249-55 (1992)) or screening for mutations by enzyme mismatch cleavage with T4 endonuclease VII (Youil et al., Proc Natl Acad Sci USA 92:87-91 (1995)) and the like. Hybridization in situ can also be used.

The DNAs or DNA fragments according to the invention can be used also for achieving the expression of human VNO pheromone receptors for drug discovery purposes and the like.

Plasmids are DNA molecules that can be introduced or transfected into cells by techniques well known in the art and which may be replicated either autonomously or after integration into the genome of the host cell. The primary expression construct used in the present invention comprises (i) a marker gene and (ii) control sequences operably linked to the marker gene and sufficient for effecting expression of the marker gene in competent cells, wherein the control sequences comprise at least one control sequence from a gene which is substantially expressed only in the competent cells. Preferably the cells are germline-competent, i.e., a cell which can contribute to germline tissue when transgenic animals are desired. Typically, the control sequences sufficient for effecting expression of the marker gene in competent cells may comprise multiple control sequences, such as a combination of a promoter and an enhancer. Alternatively, in other embodiments, a single control sequence, such as a promoter, may be sufficient for effecting expression of the marker gene in competent cells. The control sequence which is from a gene that is substantially expressed only in cells which are competent may optionally comprise an enhancer, a promoter, or both. Alternatively, the control sequences of the expression construct may comprise a negative regulatory element which is active only when a cell is differentiated. As an additional alternative, the coding sequence may encode an antisense RNA molecule and could inhibit the production of receptors that are involved in many disorders. The means of their action may be by encoding or directly being antisense, thereby inhibiting the production of hVNO-R1.

Cell lines that stably or transiently express a VNO gene may be engineered. The inventive VNO receptor gene sequence may be inserted into an expression plasmid comprising a selection marker and suitable regulatory elements, and transfected into a competent host cell. Following the introduction of the plasmid by methods known in the art (for example, calcium phosphate precipitation, electroporation and the like), engineered cells may be allowed to grow for 1-2 days in an enriched media, and then are switched to a selective media. The selectable marker in the novel plasmid confers resistance to the selection and allows cells to grow to form foci which in turn can be cloned and expanded into cell lines. This method may advantageously be used to engineer cell lines that express the desired VNO gene product on the cell surface, and are particularly useful in screening candidate drugs. For example, these cell lines are used to develop automated high throughput screening assays for novel compounds, agents or ligands with therapeutic utility in the treatment of psychiatric and endocrine disorders and diseases such as, but not limited to: premenstrual syndrome (PMS), anxiety and phobias, depression, alertness, sleep disorders, appetite control, blood pressure, pain, fertility, and hypothalamic-pituitary disorders and the like.

The transformed cells may provide the basis for the development of transgenic animals. Transformed germline-competent cells are introduced into an early stage embryo, e.g. a normal blastocyst. Transgenic animals are preferably a mammal, more preferably a rodent, and most preferably a mouse.

This method entails first introducing into embryonic cells an expression construct which comprises a marker gene operably linked to control sequences sufficient for effecting expression of the marker gene in germline-competent cells, wherein the control sequences comprise at least one control sequence from a gene which is substantially expressed only in cells which are germline-competent. Next, embryonic cells which express the marker gene sequence are identified. These cells (typically about one hundred or more) are then transferred to a recipient embryo, where the genome of the recipient embryo differs from that of the transferred cell(s). In a preferred embodiment, at least some of the cells which are transferred to the recipient embryo contribute to its development. In a final step, the recipient embryo is allowed to develop at least until the full gestational age is reached.

In another aspect of the invention there is provided antibodies for the receptor. Once the receptor has been expressed, isolated and characterized it would be advantageous to generate antibodies against the receptor. Antibodies to the receptor would be useful in the treatment of diseases and disorders associated with the overexpression of the receptor and to assess normal physiological function of the receptor.

Antiserum containing antibodies (polyclonal) is obtained by well-established techniques involving immunization of an animal, such as a rabbit, guinea pig, or goat, with an appropriate immunogen and obtaining antisera from the blood of the immunized animal after an appropriate waiting period.

Antibodies can also be obtained by somatic cell hybridization techniques, such antibodies being commonly referred to as monoclonal antibodies. Monoclonal antibodies may be produced according to the standard techniques of Köhler and Milstein, (Nature 265:495-497, 1975). Reviews of monoclonal antibody techniques are found in state-of-the-art reviews are provided by Birch et al., (1995) Monoclonal Antibodies: principles and application, Wiley-Liss, N.Y.; Davis (1995) Monoclonal antibody protocols, Humana Press, Totowa, N.J. Samples of an appropriate immunogen preparation are injected into an animal such as a mouse and, after a sufficient time, the animal is sacrificed and spleen cells obtained. Alternatively, the spleen cells of a non-immunized animal can be sensitized to the immunogen in vitro. The spleen cell chromosomes encoding the base sequences for the desired immunoglobins can be expressed by fusing the spleen cells, generally in the presence of a non-ionic detergent, for example, polyethylene glycol, with a myeloma cell line. The resulting cells, which include fused hybridomas, are allowed to grow in a selective medium, such as HAT-medium, and the surviving immortalized cells are grown in such medium using limiting dilution conditions. The cells are grown in a suitable container, e.g., microtiter wells, and the supernatant is screened for monoclonal antibodies having the desired specificity.

Various techniques exist for enhancing yields of monoclonal antibodies, such as injection of the hybridoma cells into the peritoneal cavity of a mammalian host, which accepts the cells, and harvesting the ascites fluid. Where an insufficient amount of the monoclonal antibody collects in the ascites fluid, the antibody is harvested from the blood of the host. Alternatively, the cell producing the desired antibody can be grown in a hollow fiber cell culture device or a spinner flask device, both of which are well known in the art. Various conventional ways exist for isolation and purification of the monoclonal antibodies from other proteins and other contaminants (see Köhler and Milstein, supra).

In another approach for the preparation of antibodies the sequence coding for antibody binding sites can be excised from the chromosome DNA and inserted into a cloning vector which can be expressed in bacteria to produce recombinant proteins having the corresponding antibody binding sites.

In general, antibodies can be purified by known techniques such as chromatography, e.g., Protein A chromatography, Protein G chromatography, DEAE chromatography, ABx chromatography, and the like, filtration, and so forth. Antibodies may be used as diagnostic tools such as to determine if an individual expresses the receptor encoded by SEQ ID No. 3, 4 or 6. This information will be useful to determine if a compound or drug which binds to the encoded receptor will have a physiological effect and/or therapeutic utility in a subject. Other uses include applying the antibodies as a therapeutic to block binding of a compound to the receptor on the cell surface or to prevent the receptor from interacting with and/or transducing a signal to its binding partners.

Also contemplated by the present invention are antisense oligonucleotides capable of binding to complementary sequences and blocking the expression of the human VNO receptor gene. The antisense oligonucleotides may be either. RNA or DNA. The length of antisense oligonucleotides depends, in part, on the target sequence, the desired specificity and the base composition. Effective antisense oligonucleotides are preferably at least 10, more preferably 15 or most preferably 20-30 bases long. Certain modified oligonucleotides of only 7 bases can be effective antisense oligonucleotides (see Wagner et al. (1996) Nature Biotechnology 14:840-844). Antisense oligonucleotides can contain the “normal” bases with standard 5′-3′ phosphodiester linkages or may contain modified (e.g., methylated) bases, modified sugars (e.g., methylated), non-ribose sugars, or various alternative linkages (e.g., phosphothioate, carbamate, peptide, alkylphosphonate, phosphorimadate, acetamidate, etc.), and/or mixtures of normal/modified bases and standard/alternative linkages. In another approach, an expression vector is contemplated that, when introduced into cells by techniques well known in the art, directs the synthesis of an antisense RNA that hybridized to complementary sequences in the cells.

The antisense oligonucleotides and antisense expression vectors may be used to modulate expression of the receptor in a cell. Reduced receptor expression may be desirable in various conditions and diseases including, but not limited to, fertility, modulation of hormone levels or when the receptor is over-expressed.

### Examples

The following preparations and examples are given to enable those skilled in the art to more clearly understand and to practice the present invention. They should not be considered as limiting the scope of the invention, but merely as being illustrative and representative thereof.

### Example 1

### Tissue Collection

Human VNO tissue specimens were collected for this purpose by a team of surgeons. The human VNO is located bilaterally in the nostrils, and has been associated, inter alia, with pheromone reception. The VNO is a small nasal organ with a central lumen and a pit opening to the nasal cavity. The VNO is a bilateral structure located supra palatial. The pit is approximately 1 to 1.5 mm in diameter and the lumen is approximately 1 to 1.5 cm deep. The lumen is lined with sensory neuroepithelia which constitute a distinct locus of pheromone receptors.

Collaborating otolaryngologists rinsed the human VNO specimens in sterile phosphate-buffered saline (PBS) immediately after resection to remove blood and other fluids. They rapidly excised extraneous tissue and snap-froze the VNO in liquid nitrogen. The frozen specimens were shipped on dry ice to the laboratory for RNA extraction. Thus, authentic VNO tissue specimens were collected under conditions that sought to minimize potential degradation of the RNA.

### Example 2

### Isolation of a mRNA from Pooled VNO Specimens

Total cellular RNA was extracted from the pooled VNO specimens using Trizol (Life Technologies). This procedure is rapid, and minimizes RNA degradation. However, any method for RNA isolation may be used.

Tissue samples were homogenized in Trizol Reagent (Life Technologies) using a glass-Teflon or power homogenizer. After incubation of the homogenized samples for 5 minutes at room temperature to permit the complete dissociation of nucleoprotein complexes, 0.2-ml chloroform was added per 1 ml Trizol Reagent. The samples were mixed vigorously and then centrifuged at 12,000×g for 15 minutes at 4° C. Centrifugation separated the biphasic mixtures into the lower red, phenol-chloroform phase and the upper colorless, aqueous phase.

The RNA was precipitated from the aqueous phase by mixing with 0.5 ml of isopropanol (for each initial milliliter of Trizol Reagent). The samples were incubated at room temperature for 10 minutes and centrifuged at 12,000×g for 10 minutes at 4° C. The supernatant was removed and the RNA pellet was washed once with 70% ethanol. The pellet was air dried and dissolved in diethyl pyrocarbonate (DEPC)-treated water. The RNA was quantitated by A₂₆₀ measurement.

### Example 3

### cDNA Synthesis for Library Construction

First-strand cDNA was prepared using SUPERSCRIPT II (RNase H⁻) Reverse Transcriptase (Life Technologies) which had been optimized for maximum yield of long cDNA products. The reaction was primed with a Not I-oligo(dT)₁₂₋₁₈ adapter-primer (Life Technologies) under conditions specified by the supplier. cDNA synthesis was primed by the oligo(dT)₁₂₋₁₈ at the 3′-poly(A) end of the mRNA; the adapter adds a Not I restriction site to the 5′-end of the first-strand cDNA. The reaction was incubated at 45° C. to melt potential secondary structures in the template mRNA. The length of first-strand cDNA that was synthesized in small pilot reaction mixtures containing [α-³²P]dCTP was determined, relative to known DNA standards, by alkaline agarose gel electrophoresis and autoradiography to test the quality and performance of the materials and conditions.

Second-strand synthesis was catalyzed by E. coli DNA polymerase I in combination with RNase H and E. coli DNA ligase at 16° C. In this procedure, RNase H introduces nicks into the RNA of the mRNA:cDNA hybrids and DNA polymerase I synthesizes second-strands by nick-translation; the low temperature reduces spurious synthesis by DNA polymerase I which has a tendency to strand-displace (rather than nick-translate) at higher temperatures. DNA ligase repairs nicks in the second-strands and improves the yield of long cDNAs. In the final step, T4 DNA polymerase fills in and blunts the ends of the double-stranded cDNA. The double-stranded cDNA was then deproteinized by organic extraction and precipitated with ethanol.

An excess of the commercially available Sal I adapter was ligated to the blunt ends of the double-stranded cDNA from the Not-oligo(dT)-primed reaction. Subsequent digestion with Not I removed the Sal I adapter from one end yielding molecules with a Sal I and a Not I end suitable for directional cloning into a vector that has been double-cut with these two enzymes. The recognition sites for Not I and Sal I are extremely rare in human DNA and thus the double-stranded cDNAs should be cut internally by these enzymes only very infrequently, if at all.

Unligated adapters, low molecular-weight cDNA (<500 base pairs), deoxynucleoside triphosphates, etc. were subsequently removed by chromatography on Sephacryl® S-500 HR prior to ligation into the vector. The >500-bp cDNA was ligated into pCMV-Sport 7.neo (Life Technologies) although any of a number of suitable vectors could be used. This vector has been developed at Life Technologies for cloning SUPERSCRIPT cDNA libraries. Among its features are a selectable marker gene for bacteria (β-lactamase), T7 and SP6 promoters flanking the multiple cloning site for synthesis of single-stranded sense and anti-sense cRNAs, a cytomegalovirus (CMV) promoter and SV40 polyadenylation signal for eukaryotic expression of directionally cloned inserts, and a selectable marker gene for eukaryotic cells (neo^{r}).

After ligation to the vector, the DNA was transformed into a highly competent strain of E. coli such as DH10B (Life Technologies). Recombinants were selected on LB agar plates for resistance to ampicillin. The library was amplified as described in Example 4 and plates prepared for colony hybridization.

### Example 4

### Amplification of Primary Library

The primary library was amplified once under semi-solid conditions. Semi-solid amplification of primary cDNA transformants minimizes representational biases that can occur during the expansion of plasmid cDNA libraries.

Media Preparation

2×LB: 20 g Tryptone, 10 g Yeast Extract, 10 g NaCl in 1,000 mls H₂O.

2×LB Glycerol (12.5%): 175 ml 2×LB, 25 ml Glycerol (100%). Filter sterilize and store for up to two months at room temperature.

Prepare 2 liters of 2×LB. Remove 200 mls of the 2×LB to make the 2×LB Glycerol. Place a large stir bar and 1.35 g SeaPrep (FMC) agarose into each of four 500-ml autoclavable bottles. Place bottles on stir plates. With the stir plate turned on, add 450 ml of 2×LB to each bottle, avoiding the formation of large clumps of agarose. Autoclave these bottles of 2×LB agarose for 30 min. Cool bottles in 37° C. water bath for approximately 2 hours until media reaches 37° C. After the media reaches 37° C., add Carbenicillin to 50 μg/ml (preferred antibiotic) or Ampicillin 200 μg/ml. Mix on stir plate.

Amplification

Briefly, 4×10⁵ to 6×10⁵ primary cDNA transformants (colonies from original library) were added to each of the autoclaved bottles of 2×LB agarose and mixed thoroughly on a stir plate for 2 minutes. The caps were tightened and the bottles placed in an ice water bath (0° C.) such that the level of water in the bath is at the same level as the upper level of media in the bottle. The bottles were incubated for 1 hour in the ice bath. The bottles were gently removed from the ice bath and the excess water wiped off the outside of the bottles. The bottle caps were loosened and the bottles placed in a gravity flow incubator set at 30° C. The bottles were incubated for 40-60 hrs without disturbance.

Cell Harvest

The contents of the bottles were poured into GSA bottles and centrifuged at 8,000 rpm for 20 minutes at room temperature (Caution: Make sure that the rotor was set at room temperature for at least two hours before adding the GSA bottles. Rotors at 4° C. will cause solidification of agar.) The supernatant was decanted off and the cells resuspended in a total volume of 100 ml 2×LB Glycerol (12.5%). Two 100 μl aliquots were removed for plating, further analysis, and colony estimate. Cells were filtered through sterile cheesecloth to remove agarose clumps if present.

Cell Storage

The cells were subdivided into small aliquots (Note: It is useful to make a number of 1 ml and 100 μl aliquots.) and stored at −70° C. Frozen cells can then be used to prepare DNA for experiments or can be further amplified in liquid at 30° C. to obtain DNA. Use 2.5×10⁹ cells per 100-ml growth medium for further expansion of library.

Amplified Library

The amplified library contains ˜3.5×10¹¹ colony-forming units (CFU) representing ˜1×10⁷ primary transformants. Inserts range from ≧300 to >3000 base pairs (bp) in length, with an average insert size of ˜1500 bp. For comparison, mRNAs in the rat V1R receptor family contain, on average, ˜915 bases in the open reading frame (ORF) and ˜230 bases in the 3′-untranslated region (UTR) (Dulac and Axel, 1995). Therefore, the inventive cDNA library will be a source of suitably sized clones for identification and characterization of numerous genes and gene fragments. We also point out that full-length cDNAs containing the precise 5′ end of the mRNA sequence, though scientifically interesting, are not essential provided that we obtain the entire full-length ORF (see below).

### Example 5

### Isolation of RNA from Individual VNO Specimens

RNA was isolated from individual VNO specimens using the Qiagen (Carlsbad, Calif.) RNeasy kit according to the manufacturer's instructions. In brief, each VNO specimen, frozen in liquid nitrogen, was ground with a disposable pestle in Lysis Buffer provided in the kit and then further processed according to the manufacturer's instructions. The RNA was treated with DNase and then eluted from the matrix with Elution Buffer provided in the kit. It was stored frozen at −80° C.

### Example 6

### RT-PCR

First-strand cDNA was prepared from RNA isolated from individual VNOs using the Roche Molecular (Indianapolis, Ind.) First-Strand cDNA Synthesis kit. In brief, an aliquot of VNO RNA was mixed with the appropriate buffer, salts, Mg²⁺, deoxynucleotide triphosphates, RNase inhibitor, random hexamer primers, and AMV reverse transcriptase (RT) as provided in the kit. The reaction mixture was incubated at 25° C. for 10 min to anneal the primers and then at 42° C. for 60 min to synthesize the cDNA. The mixture was heated at 99° C. for 5 min to inactivate the RT. The resulting cDNA was diluted with sterile H₂O and stored at −20° C. As a control, a reaction mixture without added RT was processed in parallel.

PCR primer pairs were designed based on the GenBank entry for the target sequence using readily available software such as Primers. Biosource (Foster City, Calif.) synthesized the primers on a standard fee-for-service basis. The primers were within or flanking the coding region of the target sequence. The PCR reaction contained the appropriate buffer, salts, Mg²⁺, deoxynucleotide triphosphates, primers, and either Expand High Fidelity polymerase (Roche Molecular) or Pfu polymerase (Stratagene; La Jolla, Calif.). Amplification was performed in a Perkin Elmer 9600 thermocycler. For internal primers and Expand polymerase, the conditions were: 94° C. for 2 min followed by 40 cycles of 94° C. for 15 sec, 56° C. for 30 sec, 72° C. for 45 sec, and one cycle of 72° C. for 5 min and 4° C. for 5 min. The template was female or male first-strand VNO cDNA or plasmid DNA from isolated from the female VNO cDNA library; aliquots of the minus-RT reactions were amplified in parallel as a negative control for the first-strand cDNA.

For flanking primers and Pfu polymerase, the conditions were: 94° C. for 2 min followed by 40 cycles of 94° C. for 15 sec, 54° C. for 30 sec, 72° C. for 1 min, and one cycle of 72° C. for 5 min and 4° C. for 5 min. The template was female or male VNO first-strand cDNA.

An aliquot of each reaction was analyzed on an agarose gel containing ethidium bromide. The amplicons were isolated from the gel, ligated into pGEM-T-Easy (Promega; Madison, Wis.), and transformed into competent E. coli DH10B (Life Technologies; Rockville, Md.). Transformants were selected on agar plates containing ampicillin. Colonies were screened for the presence of a plasmid with an insert of the expected size. Selected plasmids were sequenced to identify the amplicon and the encoded open reading frame.

### Example 7

### Sequencing

Sequencing of selected (full-length) clones was done by standard methods. Oligonucleotides that are complementary to the T7 and SP6 promoters in the vector were used to prime sequencing reactions from the ends of a cloned insert. Internal primers, based on newly acquired sequence data, were synthesized, as necessary, to sequence overlapping internal regions of the cloned cDNAs.

We examined the assembled sequences by computer for the presence of a potential full-length open reading frame. We used standard BLAST analysis to compare the human VNO clone to sequences in GenBank. The human hVNO-R1 (SEQ ID No. 1) clone shows homology to genes encoding the superfamily of G protein-coupled receptors and it predicts 7 transmembrane domains.

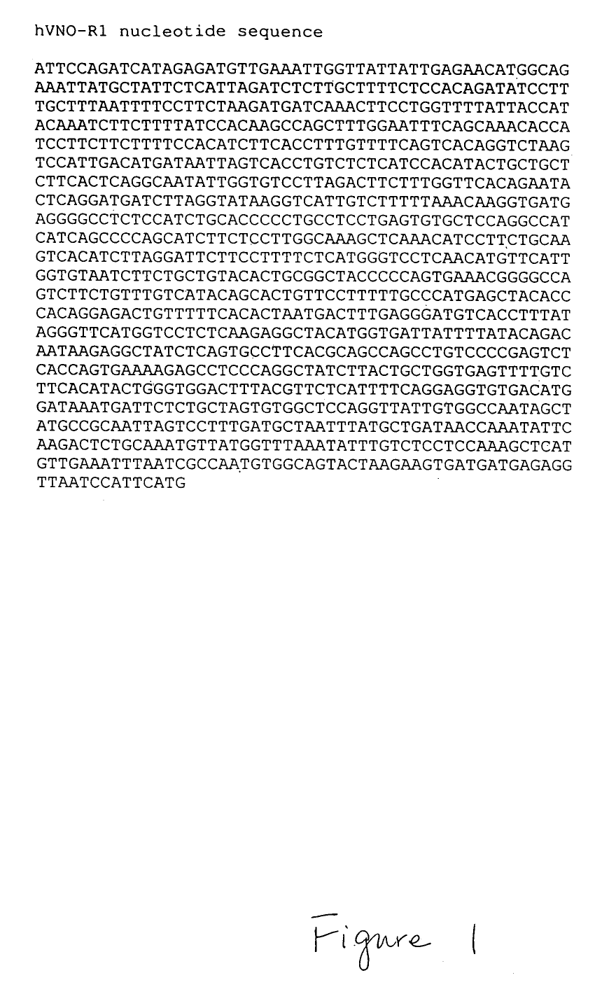

The nucleotide sequence of the hVNO-R1 cDNA (SEQ ID No. 1) is 1114 nucleotides in length containing two in-frame initiation sites at residues 17 and 44 and a termination codon at 1088. The two initiation sites yield two different forms of the receptor—a long (SEQ ID No. 3) and short (SEQ ID No. 4) form of the receptor. The initiation site located at residue 17 results in the long form of the receptor. The long form is a 357 amino acid residue protein having the amino acid sequence shown in FIG. 2 (SEQ ID No. 3). The short form has its initiation codon at residue 44 of FIG. 1. The short form is a 348 amino acid residue protein having the amino acid sequence shown in FIG. 3 (SEQ ID No. 4). The nucleic acid sequence of the short form is given in SEQ ID No. 7 or 8 (both encode the same protein).

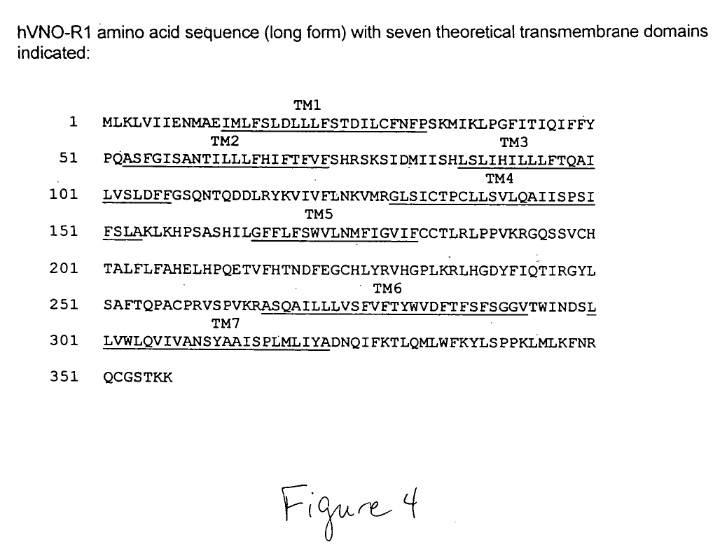

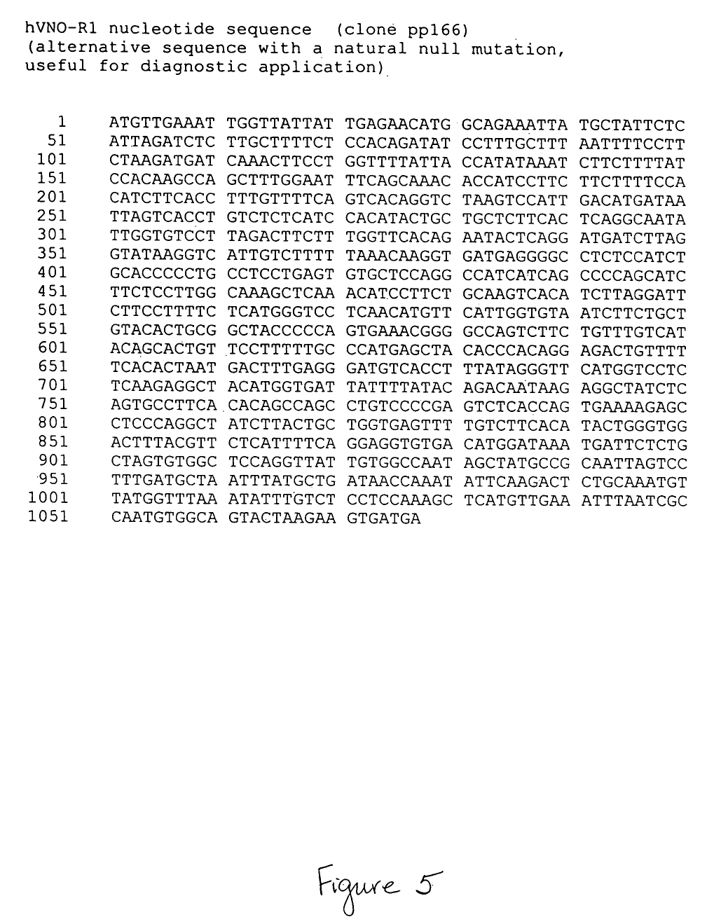

The long form of the hVNO-R1 receptor was analyzed by computer for transmembrane motifs. Similar to other G protein-coupled receptors this form was found to have 7 regions that are each likely to correspond to a transmembrane region. FIG. 4 shows the amino acid sequence of the long form of the hVNO-R1 receptor with the seven theoretical transmembrane domains underlined and indicated as TM1-TM7.

A second, mutant allele of the hVNO-R1 receptor gene was identified, mhVNO-R1 (SEQ ID No. 5). The cDNA clone begins with an initiation codon that corresponds with the first initiation codon of the hVNO-R1 clone (SEQ ID No. 1) at residue 17.

The sequence of mhVNO-R1 (SEQ ID No. 5), when aligned with hVNO-R1, is identical to hVNO-R1 except for two base differences. At residue 136 of SEQ ID No. 5 there is a C→T mutation that results in a nonsense or stop codon. Thus, translation of the mhVNO-R1 gene would result in a truncated VNO receptor protein (SEQ ID No. 6) that would be of limited functionality, if any. At position 762 of SEQ ID No. 5, mhVNO-R1 has an A residue whereas hVNO-R1 (SEQ ID No. 1) has a G at the corresponding position, i.e., position 778 of SEQ ID No. 1.

Although the foregoing invention has been described in some detail by way of illustration and example for purposes of clarity and understanding, it will be obvious that certain changes and modifications may be practiced within the scope of the appended claims.

1. An mRNA and sequences which hybridize thereto under stringent conditions that encode VNO receptor protein or fragment thereof selected from the group consisting of SEQ ID Nos. 3, 4, and 6.

2. A cDNA and sequences which hybridize thereto under stringent conditions that encode VNO receptor protein or fragment thereof selected from the group consisting of SEQ ID Nos. 3, 4, and 6.

3. A cDNA selected from the group comprising SEQ ID Nos. 1, 2, 5 and 7.

4. A cDNA and sequences which hybridize thereto under stringent conditions comprising residues 17-1088 of SEQ ID No. 1.

5. A cDNA and sequences which hybridize thereto under stringent conditions comprising residues 44-1088 of SEQ ID No. 1.

6. An expression vector comprising a cDNA and sequences which hybridize thereto under stringent conditions that encode VNO receptor protein or fragment thereof selected from the group consisting of SEQ ID Nos. 3, 4, and 6, wherein said vector is capable of expressing the encoded protein when introduced into a competent host cell.

7. An expression vector comprising a cDNA and sequences which hybridize thereto under stringent conditions comprising residues 17-1088 of SEQ ID No. 1, wherein said vector is capable of expressing the encoded protein when introduced into a competent host cell.

8. An expression vector comprising a cDNA and sequences which hybridize thereto under stringent conditions comprising residues 44-1088 of SEQ ID No. 1, wherein said vector is capable of expressing the encoded protein when introduced into a competent host cell.

9. An expression vector able to induce the synthesis of an antisense RNA which modulates expression of a receptor protein or fragment thereof selected from the group consisting of SEQ ID Nos. 3, 4, and 6.

10. A transformed cell line wherein said cell line expresses a human pheromone receptor having the amino acid sequence selected from the group consisting of SEQ ID Nos. 3, 4, and 6.

11. A transformed cell wherein said cell expresses a human pheromone receptor having the amino acid sequence selected from the group consisting of SEQ ID Nos. 3, 4, and 6.

12. The cell line of claim 10 wherein the cells are eukaryotic.

13. The cell line of claim 10 wherein the cells are prokaryotic.

14. The cell line of claim 10 wherein the cells stably express a human pheromone receptor.

15. The cell line of claim 10 wherein the cells transiently express a human pheromone receptor.

16. A protein comprising the amino acid sequence SEQ ID No. 3 and analogs thereof having about 95% homology and exhibiting VNO receptoractivity.

17. A protein comprising the amino acid sequence SEQ ID No. 4 and analogs thereof having about 95% homology and exhibiting VNO receptor activity.

18. A protein comprising the amino acid sequence SEQ ID No. 6 and analogs thereof having about 95% homology.

19. A high throughput assay comprising the cell line of claim 10 for the identification of agents that interact with a human pheromone receptor or fragment thereof selected from the group consisting of SEQ ID Nos. 3, 4 and 6.

20. A high throughput assay comprising the cell line of claim 10 for the identification of agents that interact with a human pheromone receptor or fragment thereof selected from the group consisting of SEQ ID Nos. 3, 4 and 6 wherein said ligands are useful for the treatment of depression, anxiety, phobias, blood pressure, pain, pre-menstrual syndrome, endocrine disorders, sleep disorders, alertness, and sexual desire.

21. Antibodies to a protein having an amino acid sequence selected from the group consisting of SEQ ID Nos. 3, 4 and 6.

22. An antisense oligonucleotide sequence capable of blocking the expression of hVNO-R1 (SEQ ID No. 1).

23. An antisense oligonucleotide according to claim 22 wherein said antisense oligonucleotide is between 7 and 30 residues in length.

24. A transgenic animal which functionally expresses the protein sequence selected from the group consisting of SEQ ID Nos. 3 and 4.

25. An RNA sequence and sequences which hybridize thereto under stringent conditions that encode a VNO receptor protein or fragment thereof selected from the group consisting of SEQ ID Nos. 3, 4 and 6, suitably labeled as a probe to identify a nucleic acid sequence encoding related pheromone receptors.

26. A DNA sequence and sequences which hybridize thereto under stringent conditions that encode a VNO receptor protein or fragment thereof selected from the group consisting of SEQ ID Nos. 3, 4 and 6, suitably labeled as a probe to identify a nucleic acid sequence encoding related pheromone receptors.

27. A nucleic acid sequence capable of hybridizing under low to moderate stringency conditions to at least one member of the group consisting of SEQ ID Nos. 1, 2, 5 and 7 wherein said nucleic acid sequence is suitably labeled as a probe to identify related receptor sequences and comprises at least 10 nucleic acid residues.

28. A ligand capable of interacting with a human pheromone receptor selected from the group consisting of SEQ ID Nos. 3, 4 and 6.

29. The ligand of claim 28 wherein the ligand is a naturally occurring agonist.

30. The ligand of claim 28 wherein the ligand is a synthetic agonist.

31. The ligand of claim 28 wherein the ligand is a naturally occurring antagonist.

32. The ligand of claim 28 wherein the ligand is a synthetic antagonist.

33. A therapeutically effective amount of the ligand of claim 28 wherein the ligand is useful in the treatment of depression, anxiety, phobias, blood pressure, pain, premenstrual syndrome, endocrine disorders, sleep disorders, alertness, and sexual desire.

34. A genetic test based on the difference between the two alleles of the receptor gene SEQ ID No. 1 and SEQ ID No. 5 wherein said test identifies a subject who may benefit from activation of the receptor.

35. A genetic test based on the difference between the two alleles of the receptor gene SEQ ID No. 1 and SEQ ID No. 5 wherein said test identifies a subject who may benefit from inhibition of the receptor.

36. A genetic test based on the difference between the two alleles of the receptor gene SEQ ID No. 1 and SEQ ID No. 5 wherein said test identifies a subject who may benefit from gene therapy with the expression vector comprising SEQ ID No. 1 or 2.

37. A genetic test based on the difference between the two alleles of the receptor gene SEQ ID No. 1 and SEQ ID No. 5 wherein said test identifies a subject who may benefit from gene therapy with the expression vector comprising SEQ ID No. 7.

38. Gene therapy wherein a subject is administered the expression vector comprising SEQ ID No. 1 or 2.

39. Gene therapy wherein a subject is administered the expression vector comprising SEQ ID No. 7 or 8.

40. Gene therapy wherein a subject is administered the expression vector encoding a VNO receptor protein or fragment thereof selected from the group consisting of SEQ ID Nos. 3, 4 and 6.

41. Gene therapy wherein a subject is administered the antisense expression vector of claim 9 to modulate expression of the receptor.

42. Gene therapy wherein a subject is administered the antisense oligonucleotide of claim 22 to modulate expression of the receptor.
